(19) United States
(12) Patent Application Publication
(10) Pub. No.: US 2014/0378883 A1
(43) Pub. Date: Dec. 25, 2014

(54) Garment for Promoting Dynamic Alignment of a Knee

(71) Applicant: Randall Cooper, Oakford (AU)

(72) Inventor: Randall Cooper, Oakford (AU)

(21) Appl. No.: 14/308,200

(22) Filed: Jun. 18, 2014

Related U.S. Application Data
(60) Provisional application No. 61/837,346, filed on Jun. 20, 2013.

(51) Int. Cl. A61F 5/01

(52) U.S. Cl. CPC A61F 5/0123
USPC 602/26

(57) ABSTRACT

A garment for promoting dynamic alignment of a knee of a wearer, comprising a sleeve configured to substantially conform to the knee, upper calf and lower thigh of the wearer, a strap positioned on the sleeve so as to provide a tension to promote an outward turning of the knee during flexion and extension of the knee, and wherein when the sleeve and strap are in its operative positions, a first end of the strap is attachable at a first position of the sleeve and a second end of the strap is attachable to a second position of the sleeve where the first position is medial and inferior to the patella and the second position is superior of the popliteal area behind the knee, and wherein the strap extends from the first position to the second position across the patella or tibial tuberosity and lateral aspect of the knee.

### CROSS-REFERENCE TO RELATED APPLICATIONS

The present application claims priority of U.S. Provisional Patent Application No. 61/837,346, filed Jun. 20, 2013, the content of which is incorporated herein by reference in its entirety.

### FIELD OF THE INVENTION

The present invention relates to garments for promoting dynamic alignment of a knee of a wearer. The invention also relates to methods of manufacturing these garments and use of the garments thereof.

### BACKGROUND OF INVENTION

The human knee is subjected to a wide variety of forces including compressive, bending, twisting and natural forces, particularly, if an individual is active physically or is involved in strenuous activities. Therefore, injuries to the knee are quite common along athletes and the general public.

Common injuries relate to stretching or tearing of knee ligaments, injury to the cartilage, fractures or damage to the surfaces of the knee joint. Particularly common injuries can include rupture of the Anterior Cruciate Ligament (ACL) or dislocation of the knee cap.

Recovering from these knee injuries can take many months of recovery.

Optimal knee alignment occurs during physical activity when the knee flexes in a position over the toes. However, many individuals can have knees that turn ‘inwards’ or towards the other knee during such movements as landing from a jump, pivoting, or cutting. These individuals can be at an increased risk of sustaining an Anterior Cruciate Ligament or ACL injury. This has been proven with recent research.

Other injuries of the knee include patellar subluxation or abnormal or undesirable movement of the patella. In patella subluxation, the patella can leave its normal, vertical tracking groove as a result of abnormal vector forces and/or by passive lateral or rotary forces. Weight bearing activities such as running, twisting or jumping may result in the abrupt abnormal lateral displacement of the patella from its groove which then results in immediate, temporary disability. These increased abnormal traction forces on the patella can lead to other changes of the ligaments, cartilage or tendons.

Various knee braces and knee sleeves exist which are currently used in the treatment of people with knee injuries. Some of these devices assist in relieving the load off certain weight bearing areas of the knee. Other devices may help mechanically control rotation of the knee. Basic compression sleeves exist that help enhance sensory feedback to the knee in order to aid recovery. None of these devices are designed to reduce the risk of sustaining a knee injury.

Recently, specific exercises have been proven to help reduce the chance of sustaining an ACL injury. These exercises are known as the PEP program and the FIFA 11+ program. These programs improve the strength and control of the legs and also help improve dynamic alignment of the knee during sporting movements.

However it has shown to be difficult to disseminate the information on preventative exercises to athletes and the general public as well as to encourage them to start and follow these exercise programs.

### SUMMARY OF THE INVENTION

According to an first aspect of the invention, there is provided a garment for promoting dynamic alignment of a knee of a wearer, comprising: a sleeve configured to substantially conform to the knee, upper calf and lower thigh of the wearer, a strap positioned on the sleeve so as to provide a tension to promote an outward turning of the knee during flexion and extension of the knee, and wherein when the sleeve and strap are in its operative positions, a first end of the strap is attachable at a first position of the sleeve and a second end of the strap is attachable to a second position of the sleeve where the first position is medial and inferior to the patella and the second position is superior of the popliteal area behind the knee and wherein the strap extends from the first position to the second position across the patella or tibial tuberosity and lateral aspect of the knee.

The advantage of the strap is in the ability of the strap to provide a tension from a lower medial part of the knee across the patella or tibial tuberosity and the lateral aspect of the knee to the back of the lower posterior thigh, which promotes an outward turning of the knee. During flexion and extension of the knee such as during sport or other exercise, the knee is reminded by proprioceptive and/or sensory feedback from the strap to promote an outward tinning of the knee and is discouraged from turning inward which reduces the amount of knee valgus or inward movement of the knee. The knee is also promoted and retrained towards an optical dynamic alignment where the knee is aligned with the toes or foot of the wearer during flexion and extension of the knee. This reduces the likelihood of sustaining a serious knee injury.

In a particular embodiment, the first end of the strap is permanently fixed to the first position. In particular, the first end of the strap can be sewn onto or into the sleeve so as to form a permanent attachment. Optionally, the tension of the strap can be varied in strength to suit the wearer of the sleeve.

Optionally, the second end of the strap comprises a securing device. This securing device can allow variation of the strength of the tension and can comprise a clasp, buckle or clip. In an embodiment, the securing device comprises an outer surface of a Velcro looped material at the second position of the sleeve while the strap includes an inner surface comprising a hooked material interlockable with the Velcro looped material.

Optionally, the sleeve is made of a single looped piece of fabric material. Optionally, the strap is made of a fabric material. The fabric material can allow for a garment that is breathable and easy to wear.

Optionally, the fabric material is elastic. Elastic fabric material allows the garment to provide a compressive pressure on the knee. The compressive pressure on the knee enhances the proprioceptive or sensory feedback or both to the nervous system and brain during both static and dynamic use of the garment. In an embodiment, the garment, when in its operative position, extends from above the superior pole of the patella to below the tibial tuberosity. The garment with this dimension provides the optimum feedback and dynamic alignment to the knee and is the least cumbersome.

According to a further aspect to the invention, there is provided a garment for promoting optimal tracking of a patella of a wearer during the normal range of movement of a knee, comprising: a sleeve configured to substantially conform to the knee, upper calf and lower thigh of the wearer; at least one curved support member which, when the sleeve and at least one curved support member are in their operative positions, borders the outer circumference of the patella and forms an opening, and wherein the opening substantially encircles the patella and promotes the correct tracking of the patella within the patellofemoral groove during flexion and extension of the knee.

The advantage provided by the curved support member is the promotion of the correct tracking of the patella or knee cap in the patellofemoral groove so that the risk of dislocation, subluxation or mal tracking of the patella can be reduced.

Optionally, the at least one curved support member is formed of elastic material which allows freedom of movement during flexion and extension of the knee joint.

Optionally, the at least one curved support member is integrated into or on top of the fabric of the sleeve. In an embodiment, the at least one support member comprises two arcuate shaped curved support members such that the curved support members intersect on opposite sides of the patella when the sleeve is in its operative position. In a particularly preferred embodiment, the two arcuate curved support members intersect above and below the patella.

According to an alternate aspect of the invention, there is provided a garment for promoting the stability of a knee of a wearer, comprising: a sleeve configured to substantially conform to the knee, upper calf and lower thigh of the wearer; the sleeve comprising an inner surface, at least one textured surface portion located on the inner surface adapted to stimulate activation of muscles which promote stability of the knee and wherein the textured surface portion, when the sleeve and textured surface portions are in the their operative positions, is located over or in contact with at least partially, the surface of the muscles which promote stability of the knee.

In an embodiment, the muscles comprise the quadriceps or hamstrings. In a preferred embodiment, the muscles comprise one of more of the selection of: the vastus medialis obliques (VMO), semitendinosus and semimembranosus.

Stimulation of certain muscles of the knee, in particular the VMO and semitendinosus and semimembranosus promotes stability of the knee. This further reduces the risk of any injury to the knee during strenuous activities such as jumping, twisting or kicking.

Optionally, the at least one textured surface portion comprises a plurality of raised elements. The plurality of raised elements can be formed of a silicon, rubber or polymer material. The silicon, rubber or polymer material can stimulate the surface of the leg yet is not firm enough to restrict movement, inflict pain or damage and is resilient enough to conform to the surface of the leg of the wearer. In particular, the raised elements introduce a more intense sensation to the surface of the leg, thus increasing the stimulation to the skin, muscles and nerves, in comparison to normal compressive sleeves.

According to further aspects of the present invention, there is provided method of manufacturing according to any of the embodiments described above.

According to yet further aspects of the present invention, there is provided use of the garment, according to any of the embodiments described above, during exercise or strenuous activity for the prevention of injury to the knee. In an embodiment, the garment can be used with any of the exercises of the PEP or FIFA 11+ programs.

### BRIEF DESCRIPTION OF THE DRAWINGS

In order that the present invention may be readily understood, a preferred embodiment thereof will now be described by way of example only, with reference to the accompanying drawings in which:

FIGS. 1 and 2 show respectively, front and rear views of a garment for promoting dynamic alignment of a knee; and

FIGS. 3 and 4 show the garment of FIGS. 1 and 2 on a human leg.

### DESCRIPTION OF PREFERRED EMBODIMENT

The present invention describes a garment that promotes the dynamic alignment of a knee of a wearer during sport or other physical activities, otherwise known as dynamic knee alignment. The garment comprises a sleeve which conforms anatomically to the joint of a knee. The sleeve comprises a strap which encourages the knee into a position over the toes during activities that require flexion and extension of the knee. In particular, the strap introduces tension which provides proprioceptive or sensory feedback or both which encourages the knee in an outward direction. This reduces any valgus or inward turning of the knee and therefore can reduce injury to the knee, such as damage to the ACL.

The garment also promotes correct tracking of the patella with at least one curved support member that is sewn into the sleeve. The garment can include features to improve the stability of the knee such as one or more textured portions which stimulate the muscles that assist in supporting the knee such as the quadriceps or hamstrings, and in particular, the VMO, semitendinosus or semimembranosus.

Therefore, the present invention is envisaged to be used during sport or other activities for the prevention of injuries to the knee, particularly ACL injuries or patella subluxation, dislocation or mal tracking. This garment would be especially beneficial for those with knees that display a tendency for valgus or inward turning of the knee. It is particularly envisaged that for maximum benefit, this garment can be used in conjunction with a program or exercises designed to strengthen the knee in such a way as to prevent injury or lower the risk of injury. The exercises can comprise exercises from known programs such as the PEP or FIFA 11+ programs. Therefore, the present invention can also be used to assist in the re-training of a knee to operate with correct alignment during exercise or strenuous activities.

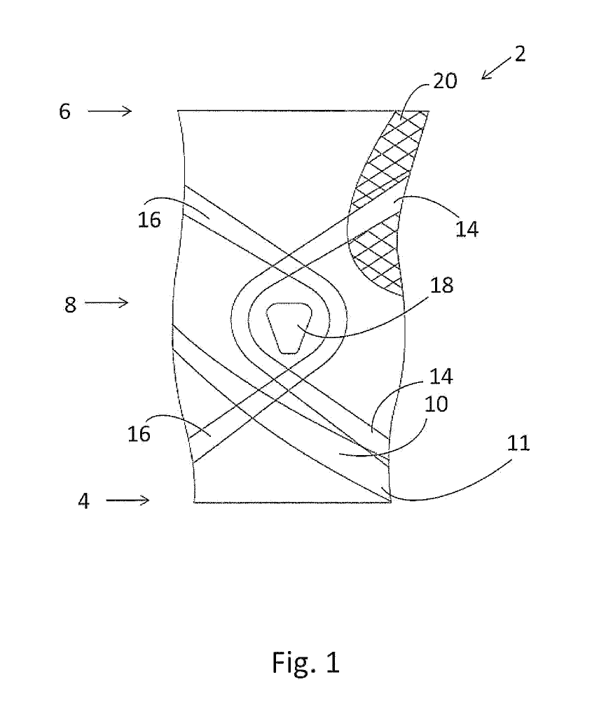

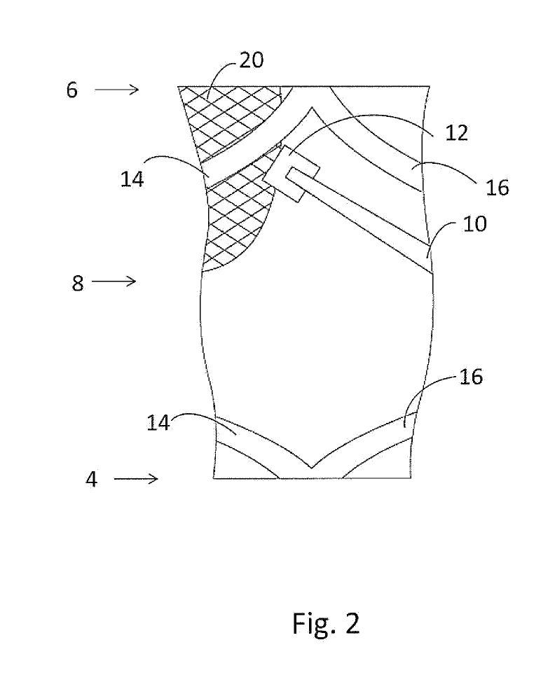

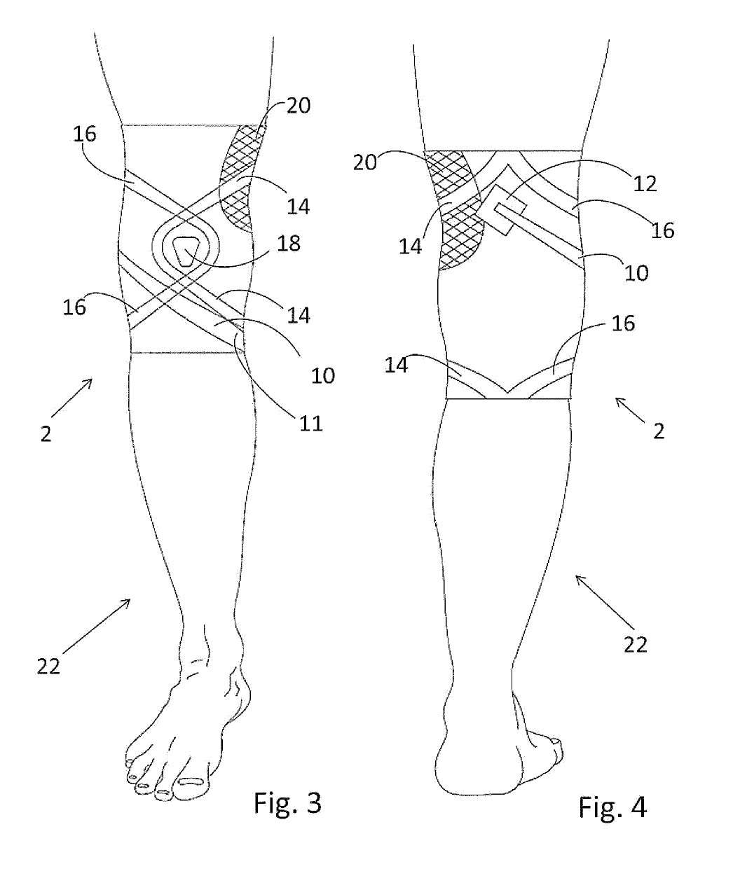

Referring now to FIGS. 1 to 4. FIGS. 1 and 2 show the garment 2 that promotes correct alignment of the knee of a wearer. In particular, FIGS. 3 and 4 show the garment 2 in its operative position on a right leg 22. The garment 2 comprises a sleeve configured to substantially conform to the knee 8, upper calf 4 and lower thigh 6 of a wearer. The garment 2 can be made of a fabric material, in particular an elastic fabric material. The garment 2 can be made of materials such as nylon, polyester, lycra, rubber, cotton, neoprene or any combination of these materials thereof. The garment 2 can also be made of a four way stretch material that provides an even compression over the knee joint. Compression of the knee joint can provide enhanced movement feedback to the nervous system and brain.

To promote alignment of the knee with the toes of the wearer during flexion and extension of the knee, a strap 10 is provided on the sleeve 2. The strap 10 extends from a first position 11 on the sleeve 2 to a second position 12 on the sleeve 2 that provides tension to promote dynamic alignment of the knee.

The strap 10 has a first end attached at a first position 11. This first position 11 is medial and inferior to the patella, that is, to say, below the knee and at a position close to the opposite leg. The strap 10 is then extendable from this first position 11, diagonally across the patella or on the tibial tuberosity and the lateral aspect of the knee until the strap 10 extends behind the knee to a second position 12 which is superior or above the popliteal area behind the knee, otherwise known as the pit of the knee. Thus, it can be seen from these Figures that the strap 10 provides a tension extending from the first position 11 to a second position 12 which encourages ‘turning out’ or outward movement of the knee. In particular, the strap provides proprioceptive or sensory feedback or both to the knee which promotes ‘turning out’ of the knee. Therefore, the strap 10 reduces the amount of knee valgus or inward movement of the knee, during flexion and extension of the knee.

The strap 10 can be made of a fabric material similar to the fabric material of the sleeve 2. In this embodiment, the strap 10 is made of a firm elastic material so that a tension can be easily applied. The first end of the strap 10, located at the first position 11 can be permanently fixed to the first position 11. However, the first end of the strap 10 can be secured by a securing device such as a clasp, buckle or clip to the first position 11.

Similarly, the second end of the strap 10, located at the second position 12, when in its operative position can be permanently fixed to the sleeve 2 by being sewn into the fabric of the sleeve 2. However, in this embodiment, the second end of the strap 10 is removable and includes a securing device, such as a clasp, buckle or clip. In a preferred embodiment, the second position 12 comprises an outer surface comprising a Velcro looped material and the second end of the strap 10 includes an inner surface comprising hooked material interlockable with the looped material. This way, if the first end of the strap 10 is permanently fixed at a first position 11, it is easy to tighten or loosen the tension across the patella or tibial tuberosity and lateral aspect of the knee and extending towards the back of the knee with an easy press on-pull off movement.

In a preferred embodiment of the invention, the sleeve 2 further comprises two curved support members, 14, 16. The curved support members 14, 16 are stitched into the sleeve 2 or on top of the sleeve 2 to assist in stability of the patella. The two curved support members 14, 16 encircle the patella to provide an opening to assist in promoting the correct tracking of the patella within the patellofemoral groove during normal flexion and extension of the knee. Therefore, the opening is of appropriate size such as to allow enough movement, during flexion and extension of the knee without restricting any movement, yet is restricted enough so as to persuade the patella from dislodging from the patellofemoral groove. The elasticity of the curved support members, formed from silicon or firm rubber tubing, allows the curved support members to flex and retract around the joint to provide the appropriate support during movement of the knee.

As shown in the figures, the curved support members 14, 16 begin at the back of the lower thigh and then run down towards the knee cap from medial to lateral or vice versa and then continue down towards the bottom of the sleeve 2 or top of the calf and around the back of the sleeve 2. The curved support members 14, 16 intersect above and below the patella.

A substantially triangular shape 18 symbolizes a marking on the sleeve 2 which can indicate to the wearer of the correct location of the patella. This ensures correct positioning of the curved supporting members 14, 16 over the knee. However this shape 18 can be any variety of shapes that indicate the intended position of the patella, such as circles, squares or other polygons.

The garment 2 can also include at least one textured portion 20 on the inner surface of the sleeve 2. In FIGS. 1 to 4, only one textured surface portion 20 is shown. The textured surface portion 20 can be used to stimulate an intense stimulation and activation of different muscles surrounding the knee. In particular, the textured surface portion 20 can be used to stimulate activation of muscles that encourage stability of the knee, such as the quadriceps or hamstrings, and specifically one or more of the muscles: VMO, semitendinosus or semimembranosus. In FIGS. 1 to 4, the textured surface portion 20 is not viewable from outside the sleeve 2. This area 20 is shown on the Figures to indicate the inner surface in contact with the muscles of the knee, in particular, the VMO, semitendinosus and semimembranosus.

The textured surface portion 20 can include a plurality of raised elements that stimulate more intense activation of these muscles. These raised elements can be formed of a silicon, rubber or polymer material. In these Figures, the textured surface portion 20 overlies the VMO, semitendinosus and semimembranosus muscles. The strap 10, curved support members 14, 16 and textured surface portion 20 can be used singly or in combination to provide a sleeve 2 that promotes dynamic and correct alignment of a knee of a wearer.

Further modifications and improvements may be made without departing from the scope of the present invention. For instance, the sleeve may be formed of any type or combination of the following materials, particularly elastic fabric material comprising cotton, nylon, polyester, neoprene, rubber, spandex or any other material known to be used in a garment for a knee or elbow joint. Similarly, the strap, curved support members, and textured surface portion can comprise silicon, rubber or a polymer material as would be known by one skilled in the art to provide the desired properties as described above.

Further, the placement or positioning of the various features of the garment such as the strap, textured surface portion or curved support members is variable such as to produce the results so described in the specification.

Additionally, a further example of modification or improvements of the garment include that the first or second end of the strap can include securing devices such as buckles, clips or clasps that allow for easy fitting, release or tightening of the tension of the strap yet do not interfere with the knee's normal movement during sport or other activities.

The garment itself can be made of a single loop of fabric material, preferably elastic fabric material. However, the garment can also be comprised of a piece of material that can be fastened around the knee joints by any sort of fasteners such as zips, buckles or additional straps.

Comprises/comprising and grammatical variations thereof when used in this specification are to be taken to specify the presence of stated features, integers, steps or components or groups thereof but do not preclude the presence or addition of one or more other features, integers, steps, components or groups thereof.

1. A garment for promoting dynamic alignment of a knee of a wearer, comprising:
a sleeve configured to substantially conform to the knee, upper calf and lower thigh of the wearer,
a strap positioned on the sleeve so as to provide a tension to promote an outward turning of the knee during flexion and extension of the knee, and
wherein, when the sleeve and strap are in its operative positions, a first end of the strap is attachable at a first position of the sleeve and a second end of the strap is attachable to a second position of the sleeve where the first position is medial and inferior to the patella and the second position is superior of the popliteal area behind the knee, and
the strap extends from the first position to the second position across the patella or tibial tuberosity and lateral aspect of the knee.

2. The garment of claim 1, wherein the strap, when in its operative position, provides proprioceptive or sensory feedback to promote an outward turning of the knee.

3. The garment of claim 1, wherein the second end of the strap comprises a securing device, which is selected from a hooked material interlockable with a looped material provided at the second position, a clasp, a buckle, or a clip.

4. The garment of claim 1, wherein the sleeve is formed from a single looped piece of fabric material.

5. The garment of claim 1, wherein the sleeve, when in its operative position, provides a compressive pressure on the knee and extends from above the superior pole of the patella to below the tibial tuberosity.

6. A garment for promoting optimal tracking of a patella of a wearer during the normal range of movement of a knee, comprising:
a sleeve configured to substantially conform to the knee, upper calf and lower thigh of a wearer;
at least one curved support member which, when the sleeve and at least one curved support member are in their operative positions, borders the outer circumference of the patella and forms an opening, and
wherein the opening substantially encircles the patella and promotes the correct tracking of the patella within the patellofemoral groove during flexion and extension of the knee.

7. The garment of claim 6, wherein the at least one support member is formed of an elastic material and is integrated into or on top of the fabric of the sleeve.

8. The garment of claim 6, comprising two arcuate shaped support members such that the support members, when in its operative position, intersect on opposite sides of the patella when the sleeve is in an operative position.

9. A garment for promoting the stability of a knee of a wearer, comprising:
a sleeve configured to substantially conform to the knee, upper calf and lower thigh of the wearer,
the sleeve further comprising an inner surface,
at least one textured surface portion located on the inner surface adapted to stimulate activation of the muscles which promote stability of the knee, and wherein the at least one textured surface portion, when the sleeve and the at least one textured surface portions are in their operative positions, is located at least partially over or in contact with the surface of the muscles which promote stability of the knee.

10. The garment of claim 9, wherein the muscles comprise vastus medialis obliques (VMO), semitendinosus, semimembranosus, or any other quadriceps or hamstring muscles.

11. The garment of claim 9, wherein the at least one textured surface portion comprises a plurality of raised elements.

12. A method of manufacturing a garment for promoting dynamic alignment of a knee of a wearer comprising the steps of:
providing a sleeve configured to substantially conform to the knee, upper calf and lower thigh of the wearer,
positioning a strap on the sleeve so as to provide a tension to promote an outward turning of the knee during flexion and extension of the knee, and
wherein, when the sleeve and strap are in their operative positions, a first end of the strap is attachable at a first position of the knee and providing a second end of the strap to be attachable at a second position of the sleeve where the first position is medial and inferior to the patella and the second position is superior of the popliteal area behind the knee, and
the strap extends from the first position to the second position across the patella or tibial tuberosity and lateral aspect of the knee.

13. The method of claim 12, further comprising permanently fixing the first end of the strap to the second position.

14. The method of claim 12, further comprising that the second end of the strap comprises a securing device, which is selected from a hooked material interlockable with a looped material provided at the second position, a clasp, a buckle, or a clip.

15. The method of claim 12, comprising forming the sleeve from a single looped piece of fabric material.

16. A method of manufacturing a garment for promoting optimal tracking of a patella of a wearer during the normal range of movement of a knee, comprising the steps of:
providing a sleeve configured to substantially conform to the knee, upper calf and lower thigh of a wearer;
providing at least one curved support member which, when the sleeve and at least one curved support member are in their operative positions, borders the outer circumference of the patella and forms an opening, and
wherein the opening substantially encircles the patella and promotes correct tracking of the patella within the patellofemoral groove during normal flexion and extension of the knee.

17. The method of claim 16, further comprising forming the at least one curved support member from an elastic material and integrating the at least one curved support member into or on top of fabric of the sleeve.

18. The method of claim 16, wherein the providing at least one curved support member comprises providing two arcuate shaped support members such that the support members intersect on opposite sides of the patella when the sleeve is in its operative position.

19. A method of manufacturing a garment for promoting the stability of a knee of a wearer, comprising the steps of:
providing a sleeve configured to substantially conform to the knee, upper calf and lower thigh of the wearer,
providing that the sleeve comprises an inner surface,
providing at least one textured surface portion located on the inner surface adapted to stimulate activation of muscles which promote stability of the knee, and
wherein, when the sleeve and the at least one textured surface portion are in their operative portions, are located at least partially over or in contact with the surface of the muscles which promote stability of the knee.

20. The method of claim 19, wherein the at least one textured surface portion comprises a plurality of raised elements.
